(19) United States
(12) Patent Application Publication
(10) Pub. No.: US 2024/0058385 A1
(43) Pub. Date: Feb. 22, 2024

(54) ANTI-VIRAL COMPOSITIONS AND METHODS

(71) Applicant: Core Consumer Healthcare LLC, Somerville, NJ (US)

(72) Inventor: Dennis O'Donnell, Basking Ridge, NJ (US)

(21) Appl. No.: 17/884,108

(22) Filed: Aug. 9, 2022

(51) Int. Cl. A61K 35/20; A61K 9/00; A61K 31/14; A61K 36/03; A61P 31/14

(52) U.S. Cl. CPC A61K 35/20; A61K 9/0073; A61K 31/14; A61K 36/03; A61P 31/14

(57) ABSTRACT

The present disclosure relates to compositions and methods for treating viral infections, or inhibiting, slowing or halting replication of a virus, e.g., an airborne respiratory virus, by administering to a nasal cavity of a subject in need thereof a composition comprising colostrum. The compositions can further comprise an algan polysaccharide, for example fucoidan. The compositions can be in the form of a nasal spray, nasal drops, nasal mist, or a nasal swab.

### FIELD

The present disclosure relates to compositions and methods for treating viral infections, or for inhibiting, slowing or halting replication of a virus, e.g., an airborne respiratory virus, by administering a composition comprising colostrum to the nasal passage.

### BACKGROUND

Respiratory viruses are prevalent causative agents of disease in humans, especially in children, with significant impact on morbidity and mortality worldwide. Human respiratory viruses include a broad range of viruses, including influenza virus, rhinovirus, respiratory syncytial virus, adenovirus, parainfluenza virus and coronaviruses. These viruses infect cells of the respiratory tract and are transmitted mainly by respiratory secretions of infected persons.

### SUMMARY OF THE INVENTION

The present disclosure relates to compositions and methods for treating viral infections, by administering to a nasal cavity of a subject in need thereof a composition comprising colostrum. The compositions can further comprise an algan polysaccharide, for example fucoidan. The compositions can be in the form of a nasal spray, nasal drops, nasal mist or nasal swab.

In some embodiments, the present disclosure relates to a method for inhibiting, halting, or slowing replication of a virus, comprising administering to a nasal cavity of a subject in need thereof a composition comprising colostrum. In some embodiments, the colostrum is a bovine colostrum.

In some embodiments, the virus is a respiratory virus. In some embodiments, the virus is an airborne respiratory virus.

In some embodiments, the virus is a non-enveloped virus. In some embodiments, the non-enveloped virus is selected from the group consisting of rhinovirus, human rhinovirus (HRV), viruses of the family picornaviridae, viruses of the family adenoiridae, adenovirus, adeno-associated virus, parvovirus, bocavirus (BoV), and human bocavirus (HBoV).

In some embodiments, the virus is an enveloped virus. In some embodiments, the enveloped virus is selected from the group consisting of influenza virus, orthomyxoviruses, coronavirus, human coronavirus (HCoV), severe acute respiratory syndrome (SARS) coronavirus (SARS-CoV), SARS-CoV-2 (COVID-19), viruses of the family paramyxoviridae, respiratory syncytial virus (RSV), human respiratory syncytial virus (HRSV)), parainfluenza virus (PIV), human parainfluenza virus (HPIV), pneumovirus (PMV), human pneumovirus (HPMV), metapneumovirus (MPV), human metapneumovirus (HMPV)), respirovirus, and rubulavirus.

In some embodiments, the composition comprises from about 0.01% to about 10% by weight of colostrum.

In some embodiments, the composition further comprises at least one excipient capable of creating an ionic charge. In some embodiments, the excipient is an organic quaternary ammonium compound that creates an electrostatic field. In some embodiments, the organic quaternary ammonium compound is benzalkonium chloride. In some embodiments, the organic quaternary ammonium compound is docosyltrimethylammonium chloride. In some embodiments, the organic quaternary ammonium compound is a Merquat copolymer.

In some embodiments, the composition further comprises at least one additional excipient selected from the group consisting of a solvent, a thickening agent, a plasticizer, a preservative, an absorption or permeability enhancing agent, a conditioner, an emulsifier, a surfactant, an antioxidant, a buffering agent, an emollient, a humectant, a suspending agent, an opacifier, a propellant, and a binder.

In some embodiments, the composition further comprises algae or an extract from algae. In some embodiments, the algae are seaweed algae selected from the group consisting of brown algae, red algae and green algae.

In some embodiments, the composition further comprises an algan polysaccharide. In some embodiments, the algan polysaccharide is derived or isolated from seaweed algae, for example brown algae, red algae or green algae. In some embodiments, the algan polysaccharide is a sulphated algan polysaccharide. In some embodiments, the algan polysaccharide is selected from the group consisting of fucoidan derived or isolated from brown algae, carrageenan derived or isolated from red algae, and ulvan derived or isolated from green algae. In some embodiments, the algan polysaccharide is a fucoidan derived or isolated from brown algae selected from the group consisting of Analipus, Chorda, Dictoyta, Fucus, Kjellmaniella, Pelvetia, Sargassum, and Undaria.

In some embodiments, the composition comprises from about 0.01% to about 10% by weight of the algan polysaccharide.

In some embodiments, the composition is at least 90% effective at killing virus particles. In some embodiments, the composition is at least 99% effective at killing virus particles.

In some embodiments, the subject has a viral infection selected from the group consisting of upper respiratory infection (URI), bronchiolitis, croup, bronchitis, pneumonia, asthma, asthma exacerbation, chronic obstructive pulmonary disease (COPD), COPD exacerbation, pharyngoconjunctival fever (PCF), and severe acute respiratory syndrome (SARS).

In some embodiments, the present disclosure provides a method for treating a viral infection, comprising administering to a nasal cavity of a subject in need thereof a composition comprising colostrum.

In some embodiments, the present disclosure provides a method for treating a condition selected from the group consisting of upper respiratory infection (URI), bronchiolitis, croup, bronchitis, pneumonia, asthma, asthma exacerbation, chronic obstructive pulmonary disease (COPD), COPD exacerbation, pharyngoconjunctival fever (PCF), and severe acute respiratory syndrome (SARS), comprising administering to a nasal cavity of a subject in need thereof a composition comprising colostrum. In some embodiments, the condition is caused by a viral infection.

In some embodiments, the viral infection is caused by a respiratory virus. In some embodiments, the viral infection is caused by an airborne respiratory virus. In some embodiments, the composition inhibits, halts or slows replication of the airborne respiratory virus. In some embodiments, the virus is a non-enveloped virus. In some embodiments, the virus is an enveloped virus.

In some embodiments, the composition is in the form of a nasal spray, nasal drops, nasal mist or nasal swab. In some embodiments, the composition further comprises an algan polysaccharide.

### DETAILED DESCRIPTION

The present disclosure relates to compositions and methods for preventing and/or treating viral infections, or for inhibiting, slowing or halting replication of a virus, e.g., an airborne respiratory virus, by administering to a nasal cavity of a subject in need thereof a composition comprising colostrum. In some embodiments, the composition is in the form of a nasal spray. In some embodiments, the composition is in the form of nasal drops. In some embodiments, the composition is in the form of a nasal mist. In some embodiments, the composition is in the form of a nasal swab. The compositions may further comprise a seaweed alga, a seaweed alga extract, or an algan polysaccharide derived from seaweed, for example a fucoidan. In some embodiments, compositions of the present disclosure comprise one or more excipients that create an electrostatic field in the area around or in the nose. The electrostatic field can reduce the inhalation of fine and/or ultra-fine airborne pollutants and, therefore, alleviate the adverse health effects such pollutants cause.

### Colostrum

In some embodiments, compositions of the present disclosure comprise colostrum as an active ingredient. In some embodiments, compositions of the present disclosure are in the form of a nasal spray, nasal drops, nasal mist or nasal swab, comprising colostrum as an active ingredient. In some embodiments, colostrum is a bovine colostrum. In some embodiments, colostrum is a human colostrum. In some embodiments, the composition comprises lactoferrin derived or isolated from bovine colostrum. In some embodiments, the composition comprises lactoferrin derived or isolated from human colostrum. In some embodiments, the colostrum is a colostrum sarcodes. In some embodiments, lactoferrin is referred to herein as lactotransferrin.

Colostrum is a milk-like fluid produced by the mammary glands of mammals (including humans) immediately following delivery of a newborn. Colostrum contains antibodies to protect the newborn against disease and infection, and immune and growth factors and other bioactives that help to activate a newborn's immune system, develop gut function, and seed a healthy gut microbiome in the first few days of life. Colostrum comprises lactoferrin, growth factors including insulin-like growth factors 1 and 2 (IGF-1 and IGF-2), and antibodies including IgA, IgG and IgM.

In some embodiments, colostrum can be present in a pharmaceutical composition as an extract. In some embodiments, colostrum can be present in a pharmaceutical composition as a purified extract. In some embodiments, colostrum can be present in a pharmaceutical composition in unpurified (i.e., natural) form. In some embodiments, a colostrum can be present in a pharmaceutical composition as an extract comprising one or more components of colostrum (e.g., lactoferrin). In some embodiments, compositions of the present disclosure comprise lactoferrin as an active ingredient. In some embodiments, compositions of the present disclosure comprise colostrum-derived lactoferrin as an active ingredient.

In some embodiments, colostrum can be present in a pharmaceutical composition comprising an extract that is devoid or substantially devoid of antibodies. In some embodiments, colostrum can be present in a pharmaceutical composition comprising an extract that is devoid or substantially devoid of colostrum-derived antibodies.

In some embodiments, colostrum can be provided as an aqueous solution, aqueous suspension, aqueous emulsion, non-aqueous solution, non-aqueous suspensions, non-aqueous emulsion, or dry powder.

In some embodiments, colostrum can be present in a pharmaceutical composition at a mass of between about 0.01% and about 10% by mass of the composition. For example, colostrum can be present in a pharmaceutical composition at a mass of between about 0.01% and about 5%, between about 0.01% and about 4%, between about 0.01% and about 3%, between about 0.01% and about 2%, between about 0.01% and about 1%, between about 0.05% and about 1%, between about 0.05% and about 0.5%, between about 0.05% and about 0.4%, between about 0.05% and about 0.3%, between about 0.05% and about 0.2%, between about 0.05% and about 0.1%, between about 0.1% and about 1%, between about 0.2% and about 1%, between about 0.3% and about 1%, between about 0.4% and about 1%, between about 0.5% and about 1% by mass of the composition.

In some embodiments, colostrum can be present at about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% by mass of the composition.

### Algan Polysaccharide

In some embodiments, compositions of the present disclosure comprise colostrum as an active ingredient, and further comprise at least one polysaccharide. In some embodiments, the at least one polysaccharide is derived or isolated from algae (i.e., an algan polysaccharide). In some embodiments, the algae are marine algae. Marine algae can be classified into three main groups based on the exhibited photosynthetic pigments: red (Rhodophyceae), brown (Phaeophyceae) and green (Chlorophycea). In some embodiments, the polysaccharide in the compositions of the present disclosure is derived or isolated from red algae (Rhodophyceae). In some embodiments, the polysaccharide in the compositions of the present disclosure is derived or isolated from brown algae (Phaeophyceae). In some embodiments, the polysaccharide in the compositions of the present disclosure is derived or isolated from green algae (Chlorophycea).

In some embodiments, compositions of the present disclosure comprise colostrum as an active ingredient, and further comprise at least one alga or alga extract, wherein the alga or alga extract comprises a polysaccharide. In some embodiments, the compositions comprise marine algae or an extract from marine algae. In some embodiments, the compositions comprise brown algae or a brown algae extract. In some embodiments, the compositions comprise red algae or a red algae extract. In some embodiments, the compositions comprise green algae or a green algae extract. In some embodiments, addition of a polysaccharide (e.g., a fucoidan), or a composition comprising a polysaccharide, can be beneficial in that it creates a physical barrier that may prevent cold and flu viruses from entering the nasal canal.

In some embodiments, the polysaccharide is a sulphated polysaccharide. Non-limiting examples of sulphated polysaccharides present in marine algae include fucoidan derived or isolated from brown algae, carrageenan derived or isolated from red algae, and ulvan derived or isolated from green algae.

In some embodiments, the sulphated polysaccharide is fucoidan. In some embodiments, the sulphated polysaccharide is fucoidan that is present in, derived from, or isolated from brown algae. The term “fucoidan” refers to complex sulphated polysaccharides mostly containing fucose residues. Aside from fucose and sulphate, fucoidan may also contain additional sugar constituents, including mannose, galactose, glucose, xylose, uronic acids and acetyl groups. There are structural similarities across different fucoidan molecules regarding the positions of inter-glycosidic linkages. Several fucoidans contain a linear backbone composed of (1→3)-linked α-L-fucose residues, while others comprise a backbone composed of alternating (1→3)- and (1→4)-linked α-L-fucose residues. Fucoidan polysaccharides may be branched. The C-2, C-3 and/or C-4 carbons of fucoidan can be substituted with sulphate groups and/or acetate groups. Non-limiting examples of brown algae include Analipus, Chorda, Dictyota, Fucus, Kjellmaniella, Pelvetia, Sargassum, and/or Undaria. In some embodiments, the fucoidan is present in, derived from or isolated from Fucus. In some embodiments, the fucoidan is present in, derived from or isolated from Fucus vesiculosus. In some embodiments, the compositions of the present disclosure comprise an extract present in, derived from or isolated from brown algae including, but not limited to, Analipus, Chorda, Dictyota, Fucus, Kjellmaniella, Pelvetia, Sargassum, and/or Undaria.

In some embodiments, the sulphated polysaccharide is carrageenan. In some embodiments, the sulphated polysaccharide is carrageenan that is present in, derived from or isolated from red algae. Carrageenan is an abundant cell wall constituent in red algae. The chemical structure is based on linear chains of repeating galactose units in d configuration (d-sugar) and 3,6-anhydro-galactose copolymer, joined by alternating α-(1→3) and β-(1→4) linkages. Examples of carrageenan include κ-, λ-, ι, μ-, θ-, β- and ν-carrageenans, all containing 15%-40% ester sulphate with the exception of β-carrageenan, which is devoid of sulphate content. Non-limiting examples of red algae include Chondrus, Euchema, Furcellaria, Gigartina, Hypnea, Iridae, and/or Kappaphycus. In some embodiments, the compositions of the present disclosure comprise an extract present in, derived from or isolated from red algae including, but not limited to, Chondrus, Euchema, Furcellaria, Gigartina, Hypnea, Iridae, and/or Kappaphycus.

In some embodiments, the sulphated polysaccharide is ulvan. In some embodiments, the sulphated polysaccharide is ulvan that is present in, isolated from, or derived from green algae. Ulvan corresponds to the major biopolymeric fraction isolated from green seaweed cell walls. Ulvan comprises sulphate, rhamnose, xylose, glucuronic acid and iduronic acid. The major repeating disaccharide comprises two different types of aldobiouronic acid, designated ulvanobiuronic acid 3-sulfate type A and type B (A3s and B3s, respectively). The A3s disaccharide is composed of glucuronic acid and sulphated rhamnose, while type B3s consists of iduronic acid and sulphated rhamnose, mainly associated via (1→4) glycosidic linkages. Rhamnose residues are sulphated mainly at position C-3 or at both positions C-2 and C-3. In some ulvan extracts, xylose or sulphated xylose residues may occur in place of uronic acids. Nonlimiting examples of green algae include Enteromorpha and Ulva. In some embodiments, the compositions of the present disclosure comprise an extract present in, derived from or isolated from green algae including, but not limited to, Enteromorpha and/or Ulva.

In some embodiments, compositions of the present disclosure comprise colostrum as an active ingredient, and further comprise at least one alga or alga extract. In some embodiments, the compositions comprise marine algae or an extract from marine algae. In some embodiments, the compositions comprise brown algae or brown algae extract. Non-limiting examples of brown algae include Analipus, Chorda, Dictyota, Fucus, Kjellmaniella, Pelvetia, Sargassum, and/or Undaria. In some embodiments, the brown alga is Fucus. In some embodiments, the brown alga is Fucus vesiculosus. In some embodiments, the compositions comprise red algae or red algae extract. Non-limiting examples of red algae include Chondrus, Euchema, Furcellaria, Gigartina, Hypnea, Iridae, and/or Kappaphycus. In some embodiments, the compositions comprise green algae or green algae extract. Non-limiting examples of green algae include Enteromorpha and/or Ulva.

An algan polysaccharide can be present in a pharmaceutical composition at a mass of between about 0.01% and about 10% by mass of the composition. For example, an algan polysaccharide can be present in a pharmaceutical composition at a mass of between about 0.01% and about 5%, between about 0.01% and about 4%, between about 0.01% and about 3%, between about 0.01% and about 2%, between about 0.01% and about 1%, between about 0.05% and about 1%, between about 0.05% and about 0.5%, between about 0.05% and about 0.4%, between about 0.05% and about 0.3%, between about 0.05% and about 0.2%, between about 0.05% and about 0.1%, between about 0.1% and about 1%, between about 0.2% and about 1%, between about 0.3% and about 1%, between about 0.4% and about 1%, between about 0.5% and about 1% by mass of the composition.

In some embodiments, an algan polysaccharide can be present at about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% by mass of the composition.

In some embodiments, algae or an algan polysaccharide can be provided as an aqueous solution, aqueous suspension, aqueous emulsion, non-aqueous solution, non-aqueous suspensions, non-aqueous emulsion, or dry powder.

### Pharmaceutical Compositions

The compounds of the present disclosure can be administered in a pharmaceutical composition suitable for administration to subjects in a biologically compatible form suitable for administration in vivo. Subjects can be, for example, adults, elderly adults, adolescents, children, toddlers, infants, and non-human animals. In some embodiments, the subject is a patient having a viral disease or is at risk of developing a viral disease.

In some embodiments, the present disclosure provides a pharmaceutical composition comprising colostrum or colostrum in combination with a sulphated polysaccharide, in admixture with a pharmaceutically-acceptable excipient. The pharmaceutically-acceptable excipient is preferably acceptable in the sense of being compatible with the other ingredients of the composition and not deleterious to the subject receiving such composition.

In some embodiments, provided herein is a composition suitable for administration to a nasal cavity. In some embodiments, provided herein is a composition in the form of a nasal spray. In some embodiments, provided herein is a composition in the form of nasal drops. In some embodiments, provided herein is a composition in the form of a nasal mist. In some embodiments, provided herein is a composition in the form of a nasal swab. In some embodiments, the pharmaceutical formulation is an aqueous solution, aqueous suspension, aqueous emulsion, non-aqueous solution, non-aqueous suspensions, non-aqueous emulsion, or dry powder.

In some embodiments, a composition of the present disclosure comprises colostrum as an active ingredient. In some embodiments, a composition of the present disclosure further comprises a sulphated polysaccharide. In some embodiments, a composition of the present disclosure further comprises at least one pharmaceutically-acceptable excipient.

In some embodiments, compositions of the present disclosure are capable of microfiltration of pollutants and particulate fine and ultra-fine size (e.g., microscopic or submicroscopic) particles that typically enter the body through the respiratory airway and/or nasal mucosa. In some embodiments, compositions of the present disclosure create an electrostatic field in the area around the nose. In some embodiments, compositions of the present disclosure comprise a quaternary compound that can create an electrostatic field in the area around the nose. The electrostatic field can reduce the inhalation of fine and/or ultra-fine airborne pollutants and, therefore, alleviate the adverse health effects such pollutants cause. Fine particle classification include, but are not limited to, pollen, dust, bacteria, mulled flour, coal dust, and asbestos. Ultra-fine particulates have a diameter of less than 0.1 micron. These include, but are not limited to, tobacco smoke, viruses, and colloidal silica.

### Quaternary Compounds

In some embodiments, the compositions of the present disclosure contain quaternary compounds, which attract oppositely-charged particles. In some embodiments, the quaternary compound is a cationic quaternary compound. In some embodiments, the compositions of the present disclosure contain cationic quaternary compounds, which repel similarly-charged particles. Therefore, when such products are applied to the skin (e.g., a nasal cavity), they reduce or prevent the inhalation/flow of airborne pollutants including but not limited to ultra-fine coal dust, yellow dust, smoke, smoke including tobacco and industrial and other airborne particulate matter to the nasal passages by filtering the pollutants outside the body before being inhaled.

In some embodiments, the cationic quaternary compound is a cationic quaternary ammonium compound. In some embodiments, the quaternary ammonium compound is an aliphatic compound. In some embodiments, the quaternary ammonium compound is a saturated compound, i.e., it contains only single bonds between atoms (e.g., carbon atoms). In some embodiments, the quaternary ammonium compound is an unsaturated compound, i.e., it contains at least one double bond (e.g., a carbon-carbon double bond). In some embodiments, the quaternary ammonium compound comprises one or more aromatic groups.

In some embodiments, the quaternary ammonium compound is a long-chain polymer which can, by way of example, contain at least 8 carbon atoms. In some embodiments, the quaternary ammonium compound comprises 9 carbon atoms. In some embodiments, the quaternary ammonium compound comprises 10 carbon atoms. In some embodiments, the quaternary ammonium compound comprises 11 carbon atoms. In some embodiments, the quaternary ammonium compound comprises 12 carbon atoms. In some embodiments, the quaternary ammonium compound comprises 13 carbon atoms. In some embodiments, the quaternary ammonium compound comprises 14 carbon atoms. In some embodiments, the quaternary ammonium compound comprises 15 carbon atoms. In some embodiments, the quaternary ammonium compound comprises 16 carbon atoms. In some embodiments, the quaternary ammonium compound comprises 17 carbon atoms. In some embodiments, the quaternary ammonium compound comprises 18 carbon atoms. In some embodiments, the quaternary ammonium compound comprises 19 carbon atoms. In some embodiments, the quaternary ammonium compound comprises 20 carbon atoms. In some embodiments, the quaternary ammonium compound comprises 21 carbon atoms. In some embodiments, the quaternary ammonium compound comprises 22 carbon atoms. In some embodiments, the quaternary ammonium compound comprises 23 carbon atoms. In some embodiments, the quaternary ammonium compound comprises 24 carbon atoms. In some embodiments, the quaternary ammonium compound comprises 25 carbon atoms. In some embodiments, the quaternary ammonium compound comprises 26 carbon atoms. In some embodiments, the quaternary ammonium compound comprises 27 carbon atoms. In some embodiments, the quaternary ammonium compound comprises 29 carbon atoms. In some embodiments, the quaternary ammonium compound comprises 30 carbon atoms. In some embodiments, the quaternary ammonium compound comprises more than 30 carbon atoms.

In some embodiments, the quaternary ammonium compound is an organic compound of the general structure N(R)₄⁺, wherein each R is independently of the others an alkyl, arylalkyl, cycloalkyl, alkenyl, alkynyl, or an aryl group.

An “alkyl” group refers to any saturated aliphatic hydrocarbon, including straight-chain and branched-chain alkyl groups. Non-limiting examples of alkyl groups include straight, branched, and cyclic alkyl groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of straight alkyl groups include methyl, ethyl, propyl, isopropyl, n-butyl, isobutyl, sec-butyl, tert-butyl, pentyl, hexyl, heptyl, octyl, nonyl, decyl, docosyl, and the like. Branched alkyl groups include any straight alkyl group substituted with any number of alkyl groups.

An “alkenyl” group refers to any unsaturated aliphatic hydrocarbon, including at least one carbon-carbon double bond. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl, and the like.

An “alkynyl” group refers to any unsaturated aliphatic hydrocarbon, including at least one carbon-carbon triple bond. Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and the like.

A “cycloalkyl” group refers to a saturated or unsaturated cyclic hydrocarbon, including monocyclic or polycyclic groups. Non-limiting examples of cyclic alkyl groups include cyclopropyl, cyclobutyl, cyclopentyl, cyclohexyl, cycloheptyl, and cyclooctyl groups. Cyclic alkyl groups also include fused-, bridged-, and spiro-bicycles and higher fused-, bridged-, and spiro-systems. Non-limiting examples of cyclic alkyl groups include cyclopropyl, cyclobutyl, cyclopentyl, cyclohexyl, cyclohepyl, cyclooctyl, and the like.

An “aryl” group refers to an aromatic carbocyclic group. An aryl group can be monocyclic or polycyclic. Non-limiting examples of aryl groups include phenyl, toluyl, naphthyl, pyrrolyl, pyridyl, imidazolyl, thiophenyl, and furyl. Non-limiting examples of substituted aryl groups include phenyl, phenyl, naphthyl including 1-naphthyl and 2-naphthyl.

An “arylalkyl” group refers to an aryl group as described herein, linked to an alkyl group described herein. An example of an arylalkyl group is a benzyl group.

A quaternary ammonium compound described herein can be provided as a pharmaceutically-acceptable salt with an appropriate counter anion of an organic or inorganic acids. Non-limiting examples of such acids include hydrochloric, hydrofluoric, hydrobromic, hydroiodic, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, and cinnamic acid.

A suitable counter anion of an organic or inorganic acid can be, for example, a halide (e.g., chloride, fluoride, bromide, iodide), trifluoroacetate, sulfate (sulphate), phosphate, acetate, succinate, citrate, lacate, maleate, fumarate, palmitate, cholate, pamoate, mucate, D-glutamate, D-camphorate, glutarate, phthalate, tartarate, laurate, stearte, salicyclate, methanesulfonate, benzenesulfonate, sorbate, picrate, benzoate, or cinnamate.

In some embodiments, the quaternary ammonium compound is a behentrimonium salt. A behentrimonium salt can be represented by the following chemical formula:

CH₃—(CH₂)₂₁—N(CH₃)₃⁺X⁻

wherein X⁻ is a counter anion, for example a counter anion of an organic or inorganic acid as described herein. In some embodiments, X⁻ is a halide. In some embodiments, X⁻ is a sulphate.

In some embodiments, the quaternary ammonium compound is a behentrimonium halide. In some embodiments, the quaternary ammonium compound is behentrimonium chloride (docosyltrimethylammonium chloride). Behentrimonium chloride is represented by the following chemical formula:

CH₃—(CH₂)₂₁—N(CH₃)₃⁺Cl⁻

In some embodiments, the quaternary ammonium compound is a benzalkonium salt. A benzalkonium salt can be represented by the following chemical formula:

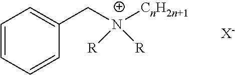

wherein
n is 8-18;
X⁻ is a counter anion, for example a counter anion of an organic or inorganic acid as described herein; and
each R is independently of the other an alkyl, arylalkyl, cycloalkyl, alkenyl, alkynyl, or an aryl group.

In some embodiments, each R is a methyl group, and the benzalkonium salt is an alkyldimethylbenzylammonium salt represented by the following chemical formula:

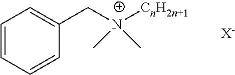

wherein n is 8-18; and
X⁻ is a counter anion, for example a counter anion of an organic or inorganic acid as described herein.

In some embodiments, X⁻ is a halide. In some embodiments, X⁻ is a sulphate. In some embodiments, n is 8, 10, 12, 14, 16 or 18. In some embodiments, n is 12-16, and the benzalkonium salt is a benzyl C12-16 alkyldimethyl ammonium salt.

In some embodiments, the quaternary ammonium compound is a benzalkonium halide. In some embodiments, the quaternary ammonium compound is a benzalkonium sulphate. In some embodiments, the quaternary ammonium compound is benzalkonium chloride.

In some embodiments, the quaternary ammonium compound is a Merquat polymer. Merquat polymers (Lubrizol) are highly charged quaternary ammonium polymers comprising 5 types of monomers: diallyldimethylammonium, methacrylamidopropyl trimethyl ammonium chloride, acrylate, acrylamide, and methyl acrylate. Examples of Merquat polymers include, but are not limited to, Merquat 100, Merquat 280, Merquat 281, Merquat 295, Merquat 550, Merquat 2001, Merquat 2003, Merquat 3300, Merquat 3331, and Merquat 2003.

In some embodiments, formulations containing a quaternary ammonium salt, e.g., Behentrimonium chloride, benzalkonium chloride and/or Merquat polymer, when applied in the vicinity of nasal passages, can prevent fine and ultra-fine particles from entering the nasal passages by creating an electrostatic field that trap these particles prior to inhalation.

A quaternary ammonium compound can be present in a pharmaceutical composition at a mass of between about 0.01% and about 10% by mass of the composition. For example, quaternary ammonium compound can be present in a pharmaceutical composition at a mass of between about 0.01% and about 5%, between about 0.01% and about 4%, between about 0.01% and about 3%, between about 0.01% and about 2%, between about 0.01% and about 1%, between about 0.05% and about 1%, between about 0.05% and about 0.5%, between about 0.05% and about 0.4%, between about 0.05% and about 0.3%, between about 0.05% and about 0.2%, between about 0.05% and about 0.1%, between about 0.1% and about 1%, between about 0.2% and about 1%, between about 0.3% and about 1%, between about 0.4% and about 1%, between about 0.5% and about 1% by mass of the composition.

In some embodiments, quaternary ammonium compound can be present at about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% by mass of the composition.

### Excipients

In some embodiments, compositions of the present disclosure (e.g., nasal spray) can be applied directly to the nasal cavity using a device. The device can be, for example, a metering atomizing spray pump, e.g., a single, bi-dose or multiuse spray device, with or without a propellant. Examples of propellants include, but are not limited to, chlorofluorocarbons, hydrochlorofluorocarbons, hydrofluorocarbons, hydrocarbons, and compressed gases. Other examples of nasal compositions include, but are not limited to, nasal drops, nasal mist or nasal swab. Compositions can be in the form of a liquid, solution, suspension or emulsion.

In some embodiments, a composition of the present disclosure (e.g., a nasal spray, nasal drops, nasal mist or nasal swab) can comprise at least one pharmaceutically-acceptable excipient. Suitable pharmaceutically-acceptable excipients include, but are not limited to, a solvent, a thickening agent, a plasticizer, a preservative, an absorption or permeability enhancing agent, a conditioner, an emulsifier, a surfactant, an antioxidant, a buffering agent, an emollient, a humectant, a suspending agent, an opacifier, and a binder.

In some embodiments, the composition (e.g., a nasal spray, nasal drops, nasal mist or nasal swab) comprises a pharmaceutically-acceptable solvent. In some embodiments, the solvent is a polar solvent. Non-limiting examples of solvents include water, linear or branched C₂ to C₁₈ alcohols, C₂ to C₈ polyalcohols, polyethyleneglycols, and combinations thereof. In some embodiments, the solvent is water. In some embodiments, the solvent is propylene glycol.

In some embodiments, the composition (e.g., a nasal spray, nasal drops, nasal mist or nasal swab) comprises a pharmaceutically-acceptable thickening agent. Non-limiting examples of pharmaceutically-acceptable thickening agents include hydroxyethyl cellulose, microcrystalline cellulose (MCC) and sodium carboxymethyl cellulose (Na-CMC).

In some embodiments, the composition (e.g., a nasal spray, nasal drops, nasal mist or nasal swab) comprises a pharmaceutically-acceptable plasticizing agent (plasticizer). Non-limiting examples of pharmaceutically-acceptable plasticizing agents include caprylic acid, capric triglyceride and triethyl citrate.

In some embodiments, the composition (e.g., a nasal spray, nasal drops, nasal mist or nasal swab) comprises a pharmaceutically-acceptable binder. Non-limiting examples of pharmaceutically-acceptable binders include microcrystalline cellulose, gum tragacanth, gum arabic, gelatin, polyvinylpyrrolidone, copovidone, hydroxypropyl methylcellulose, and starch.

In some embodiments, the composition (e.g., a nasal spray, nasal drops, nasal mist or nasal swab) comprises a pharmaceutically-acceptable surfactant. Non-limiting examples of pharmaceutically-acceptable surfactants include sodium laurylsulfate or polysorbates, polyvinyl alcohol (PVA), polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil, and hardened castor oil such as polyoxyethylene hardened castor oil.

In some embodiments, the composition (e.g., a nasal spray, nasal drops, nasal mist or nasal swab) comprises a pharmaceutically-acceptable preservative. Non-limiting examples of preservatives include benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol,

In some embodiments, the composition (e.g., a nasal spray, nasal drops, nasal mist or nasal swab) comprises an absorption or permeability enhancing agent. Non-limiting examples of pharmaceutically-acceptable absorption or permeability enhancing agent include alkylglycosides, benzalkonium chloride, oleic acid, or salt thereof, polysorbate 20, polysorbate 80, sodium lauryl sulfate, cyclodextrins, medium and long chain fatty acids, or salts thereof, saturated and unsaturated fatty acids, or salts thereof, alcohol, glycerin, propylene glycol, PEG 300/400, and benzyl alcohol.

In some embodiments, the composition (e.g., a nasal spray, nasal drops, nasal mist or nasal swab) further comprises a pharmaceutically-acceptable antioxidant. Non-limiting examples of pharmaceutically-acceptable antioxidants include alpha tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof.

In some embodiments, the composition (e.g., a nasal spray, nasal drops, nasal mist or nasal swab) further comprises a buffering agent. Buffering agents include, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.11% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.11% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, between about 0.1% and about 1% by mass of the composition.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the composition.

Suitable compositions for use in the compositions and methods described herein are disclosed in U.S. Pat. Nos. 9,750,706 and 9,737,497, the contents of each of which are hereby incorporated by reference in their entirety.

### Treatment of Viral Infections

In some embodiments, the pharmaceutical compositions provided herein are useful for treating, preventing or reducing the likelihood of occurrence of viral infections. Provided herein is method for treating a viral infection, comprising administering to a nasal cavity of a subject in need thereof a composition comprising colostrum. In some embodiments, the composition is in the form of a nasal spray, nasal drops, nasal mist or nasal swab.

The term “treating” or “treatment” as used herein refers to abrogating, inhibiting, slowing, halting, or reversing the progression of a disease or condition, ameliorating clinical symptoms of a disease or condition or preventing the appearance of clinical symptoms of a disease or condition, e.g., a viral infection.

In some embodiments, the viral infection is a respiratory viral infection. In some embodiments, the viral infection is a respiratory viral infection caused by an airborne respiratory virus. In some embodiments, the virus is a DNA virus. In some embodiments, the virus is an RNA virus. In some embodiments, the virus is a retrovirus.

In some embodiments, the pharmaceutical compositions provided herein are useful for halting, slowing, or inhibiting replication of a virus, e.g., an airborne respiratory virus. In some embodiments, the pharmaceutical compositions provided herein are useful reducing viral load, e.g., of an airborne respiratory virus. In some embodiments, provided herein is a method for halting, slowing, or inhibiting replication of a respiratory virus, comprising administering to a nasal cavity of a subject in need thereof a composition comprising colostrum. In some embodiments, the respiratory virus is an airborne respiratory virus. In some embodiments, the composition is in the form of a nasal spray, nasal drops, nasal mist or nasal swab.

In some embodiments, provided herein is a method reducing viral load of a respiratory virus, comprising administering to a nasal cavity of a subject in need thereof a composition comprising colostrum. In some embodiments, the respiratory virus is an airborne respiratory virus. In some embodiments, the composition is in the form of a nasal spray, nasal drops, nasal mist or nasal swab. In some embodiments, the virus is an enveloped virus. Examples of enveloped respiratory viruses include viruses of the family paramyxoviridae. Paramyxoviridae viruses include (i) respiratory syncytial virus (e.g., human respiratory syncytial virus (HRSV)); (ii) parainfluenza virus (e.g., human parainfluenza virus (HPIV), including HPIV-1, HPIV-2, HPIV-3, HPIV-4A, and HPIV-5A); (iii) pneumovirus (e.g., human pneumovirus (HPMV)); (iv) metapneumovirus (e.g., human metapneumovirus (HMPV)); (v) respirovirus; and (vi) rubulavirus.

Other examples of enveloped respiratory viruses include Coronaviruses. Coronaviruses include human coronaviruses (HCoV), severe acute respiratory syndrome (SARS) coronaviruses (SARS-CoV), and SARS-CoV-2 (COVID-19).

Other examples of enveloped viruses include viruses of the family Orthomyxoviridae. Orthomyxoviridae viruses include influenza viruses, such as genus Alphainfluenzavirus, Betainfluenzavirus, Gammainfluenzavirus, Deltainfluenzavirus, Isavirus, Thogotovirus, and Quaranjavirus. In some embodiments, the Orthomyxoviridae is an influenza virus. In some embodiments, the influenza virus is a genus Alphainfluenzavirus. Examples of Alphainfluenzavirus (influenza A) including subtypes H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7. In some embodiments, the influenza virus is a genus Betainfluenza (influenza B virus) including subtypes Victoria and Yamagata. In some embodiments, the influenza virus is a gammainfluenza virus (influenza C virus).

In some embodiments, the virus is a non-enveloped virus. Examples of non-enveloped respiratory viruses include viruses of the family Picornaviridae, including rhinovirus (including human rhinovirus (HRV) species A, B and C). Other examples of non-enveloped respiratory viruses include viruses of the family Adenoiridae, including adenovirus, adeno-associated viruses (e.g., parvoviruses). Other examples of non-enveloped respiratory viruses include Bocaviruses (including human bocavirus (HBoV).

In some embodiments, the viral infection is leads to or is associated with a disease, condition or syndrome. In some embodiments, the disease, condition or syndrome is an upper respiratory infection (URI). In some embodiments, the disease, condition or syndrome is bronchiolitis. In some embodiments, the disease, condition or syndrome is croup. In some embodiments, the disease, condition or syndrome is bronchitis. In some embodiments, the disease, condition or syndrome is pneumonia. In some embodiments, the disease, condition or syndrome is asthma. In some embodiments, the disease, condition or syndrome is asthma exacerbations. In some embodiments, the disease, condition or syndrome is chronic obstructive pulmonary disease (COPD). In some embodiments, the disease, condition or syndrome is COPD exacerbations. In some embodiments, the disease, condition or syndrome is pharyngoconjunctival fever (PCF). In some embodiments, the disease, condition or syndrome is severe acute respiratory syndrome (SARS).

In some embodiments, a composition comprising colostrum is effective at treating a viral infection, for example a viral infection caused by a non-enveloped virus, e.g., an airborne respiratory non-enveloped virus. In some embodiments, a composition comprising colostrum is effective at treating a viral infection, for example a viral infection caused by an enveloped virus, e.g., an airborne respiratory enveloped virus.

In some embodiments, a composition comprising colostrum is effective at reducing or eliminating viral load. In some embodiments, a composition comprising colostrum is effective at inhibiting replication of a virus, e.g., an airborne respiratory virus. In some embodiments, a composition comprising colostrum is effective at halting replication of a virus, e.g., an airborne respiratory virus. In some embodiments, a composition comprising colostrum is effective at slowing replication of a virus, e.g., an airborne respiratory virus. In some embodiments, a composition comprising colostrum is effective at reducing viral load of an airborne respiratory virus.

In some embodiments, a composition comprising colostrum is effective at killing virus particles. In some embodiments, the composition is at least 50% effective at reducing viral load and/or killing virus particles. In some embodiments, the composition is at least 55% effective at reducing viral load and/or killing virus particles. In some embodiments, the composition is at least 60% effective at reducing viral load and/or killing virus particles. In some embodiments, the composition is at least 65% effective at reducing viral load and/or killing virus particles. In some embodiments, the composition is at least 70% effective at reducing viral load and/or killing virus particles. In some embodiments, the composition is at least 75% effective at reducing viral load and/or killing virus particles. In some embodiments, the composition is at least 80% effective at reducing viral load and/or killing virus particles. In some embodiments, the composition is at least 85% effective at reducing viral load and/or killing virus particles. In some embodiments, the composition is at least 90% effective at reducing viral load and/or killing virus particles. In some embodiments, the composition is at least 95% effective at reducing viral load and/or killing virus particles. In some embodiments, the composition is at least 96% effective at composition is at least 97% effective at reducing viral load and/or killing virus particles. In some embodiments, the composition is at least 98% effective at reducing viral load and/or killing virus particles. In some embodiments, the composition is at least 99% effective at reducing viral load and/or killing virus particles. In some embodiments, the composition is at least 99.1% effective at reducing viral load and/or killing virus particles. In some embodiments, the composition is at least 99.2% effective at reducing viral load and/or killing virus particles. In some embodiments, the composition is at least 99.3% effective at reducing viral load and/or killing virus particles. In some embodiments, the composition is at least 99.4% effective at reducing viral load and/or killing virus particles. In some embodiments, the composition is at least 99.5% effective at reducing viral load and/or killing virus particles. In some embodiments, the composition is at least 99.6% effective at reducing viral load and/or killing virus particles. In some embodiments, the composition is at least 99.7% effective at reducing viral load and/or killing virus particles. In some embodiments, the composition is at least 99.8% effective at reducing viral load and/or killing virus particles. In some embodiments, the composition is at least 99.9% effective at reducing viral load and/or killing virus particles. In some embodiments, the composition is 100% effective at reducing viral load and/or killing virus particles.

In some embodiments, a composition comprising colostrum and an algan polysaccharide is effective at treating a viral infection, for example a viral infection caused by a non-enveloped virus. In some embodiments, a composition comprising colostrum and an algan polysaccharide is effective at treating a viral infection, for example a viral infection caused by an enveloped virus. In some embodiments, a composition comprising colostrum and an algan polysaccharide is effective at reducing or eliminating viral load. In some embodiments, a composition comprising colostrum and an algan polysaccharide is effective at killing virus particles. In some embodiments, the composition is at least 50% effective at reducing viral load and/or killing virus particles. In some embodiments, the composition is at least 55% effective at reducing viral load and/or killing virus particles. In some embodiments, the composition is at least 60% effective at reducing viral load and/or killing virus particles. In some embodiments, the composition is at least 65% effective at composition is at least 70% effective at reducing viral load and/or killing virus particles. In some embodiments, the composition is at least 75% effective at reducing viral load and/or killing virus particles. In some embodiments, the composition is at least 80% effective at reducing viral load and/or killing virus particles. In some embodiments, the composition is at least 85% effective at reducing viral load and/or killing virus particles. In some embodiments, the composition is at least 90% effective at reducing viral load and/or killing virus particles. In some embodiments, the composition is at least 95% effective at reducing viral load and/or killing virus particles. In some embodiments, the composition is at least 96% effective at reducing viral load and/or killing virus particles. In some embodiments, the composition is at least 97% effective at reducing viral load and/or killing virus particles. In some embodiments, the composition is at least 98% effective at reducing viral load and/or killing virus particles. In some embodiments, the composition is at least 99% effective at reducing viral load and/or killing virus particles. In some embodiments, the composition is at least 99.1% effective at reducing viral load and/or killing virus particles. In some embodiments, the composition is at least 99.2% effective at reducing viral load and/or killing virus particles. In some embodiments, the composition is at least 99.3% effective at reducing viral load and/or killing virus particles. In some embodiments, the composition is at least 99.4% effective at reducing viral load and/or killing virus particles. In some embodiments, the composition is at least 99.5% effective at reducing viral load and/or killing virus particles. In some embodiments, the composition is at least 99.6% effective at reducing viral load and/or killing virus particles. In some embodiments, the composition is at least 99.7% effective at reducing viral load and/or killing virus particles. In some embodiments, the composition is at least 99.8% effective at reducing viral load and/or killing virus particles. In some embodiments, the composition is at least 99.9% effective at reducing viral load and/or killing virus particles. In some embodiments, the composition is 100% effective at reducing viral load and/or killing virus particles.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

### EXAMPLES

The following are examples that illustrate embodiments for practicing the disclosure described herein. These examples should not be construed as limiting.

### Example 1: Materials and Methods

A formulation according to the present disclosure is provided in Table 1.

TABLE 1
Formulation 1

| Ingredient | Function |
|---|---|
| Colostrum sarcodes 3X (0.1%) | Active |
| Purified Water USP | Solvent |
| Propylene Glycol | Solvent |
| Viviapur MCG 591P (microcrystalline | Thickening agent |
| cellulose) | |
| Aloe Vera 10x Concentrate | Moisturizer |
| Rose Water | Flavoring agent |
| Potassium Sorbate | Preservative |
| SP Versa Flex V-150 | Emulsifier |
| Lonzagard BKC cGMP (benzalkonium | Cationic agent |
| chloride) | |
| Monobasic Phosphate Sodium | Buffer |
| (Anhydrous) | |
| Dibasic Phosphate Sodium (Anhydrous) | Buffer |
| Merquat 295 Polymer | Cationic agent |
| Menthol | Coating agent/fragrance |
| Incroquat Behenyl TMC-85-PA | Conditioner, emulsifier, |
| | cationic agent |

A formulation according to the present disclosure is provided in Table 2.

TABLE 2
Formulation 2

| Ingredient | Function |
|---|---|
| Colostrum sarcodes 3X (0.1%) | Active |
| Fucus vesiculosus 3X (0.1%) | Active |
| Purified Water USP | Solvent |
| Propylene Glycol | Solvent |
| Viviapur MCG 591P (microcrystalline | Thickening agent |
| cellulose) | |
| Aloe Vera 10x Concentrate | Moisturizer |
| Rose Water | Flavoring agent |
| Potassium Sorbate | Preservative |
| SP Versa Flex V-150 | Emulsifier |
| Lonzagard BKC cGMP (benzalkonium | Cationic agent |
| chloride) | |
| Monobasic Phosphate Sodium | Buffer |
| (Anhydrous) | |
| Dibasic Phosphate Sodium (Anhydrous) | Buffer |
| Merquat 295 Polymer | Cationic agent |
| Menthol | Coating agent/fragrance |
| Incroquat Behenyl TMC-85-PA | Conditioner, emulsifier, |
| | cationic agent |

### Example 2: Antiviral Activity

### Materials and Methods

Antiviral activity of three formulations as set forth in Table 3 was evaluated.

TABLE 3

| Formulation | Ingredients |
|---|---|
| 1 | Fucoidan (0.1 wt %) with |
| | benzalkonium chloride (BKC) (0.1 |
| | wt %) |
| 2 | Bovine colostrum Lactoferrin (0.1 |
| | wt %) with BKC (0.1 wt %) |
| 3 | Combination of two active |
| | ingredients (each at 0.1 wt %) with |
| | BKC (0.1 wt %) |

The study used ASTM method E1052 “Efficacy of Antimicrobial Agents Against Viruses in Suspension” to assess the antimicrobial efficacy against two bacteriophages: enveloped (φ174) and non-enveloped (MS2). The φX174 and MS2 bacteriophage systems are designed to test antiviral activity against enveloped and non-enveloped viruses, respectively. Generally, the method comprises the following steps: the virus is exposed to the virucide for a defined period of time. After the appropriate exposure interval, the mixture is evaluated for viable virus in the appropriate host system.

The following bacteriophage ATTC strains were used:
MS2 Source: ATCC #15597-B1
φX174 Source: ATCC #13706-B1

Bacteriophages are viruses capable of infecting bacterial cells. MS2 is a bacteriophage model used to study antiviral activity of non-enveloped viruses. MS2 infects “male” Eschericia coli, which are bacterial cells capable of passing a portion of their genetic material (typically in plasmid form) to other bacterial cells through a structure called a pilus. Morphologically, MS2 is a non-enveloped, icosahedral virus. MSA bacteriophage is suitable as a screening tool for anti-viral activity as it requires a bacterial host (which replicates rapidly) instead of mammalian host cells (which replicate slowly). Anti-viral screening with MS2 allows to test a large number of formulations and/or evaluate multiple contact times. MS2 virions are 23-28 nm in diameter, putting them in the category of small non-enveloped viruses. MS2 is generally more resistant to inactivation than enveloped viruses but is more sensitive to inactivation than other small non-enveloped viruses to various methods of disinfection, including UVC light exposure and quaternary ammonium compounds.

Bacteriophage φX174 is a small icosahedral virus that contains a single-stranded, closed circular DNA molecule, and that infects Escherichia coli. φX174 is a bacteriophage model used to study antiviral activity of enveloped viruses. (A. P. Roznowski, S. M. Doore, B. A. Fane, Phi ((φ))X174, Genetics of, Reference Module in Life Sciences, Elsevier, 2017)

The three formulations of Table 3 were evaluated against φX174 and MS2. One sample was prepared per formulation, and three technical replicates were run for each sample, in a blinded mode. Bacteriophages were exposed to the three formulations for 15 minutes, and plaque assays were used to measure viral loads and reduction.

### Sample Preparation

Samples were weighed and resuspended in benzalkonium chloride 0.1% prepared with sterile deionized water. The concentrations prepared were the following:
Fucoidan (0.1 wt %) with benzalkonium chloride (0.1 wt %)
Bovine colostrum ferritin (0.1 wt %) with benzalkonium chloride (0.1 wt %)
Combination of both active ingredients (each at 0.1 wt %) with benzalkonium chloride (0.1 wt %)

Sample sterility was confirmed by plating prior to ASTM E1052 testing.

### Method E1052

ASTM method E1052 was performed in a blinded mode as follows:
a) Bacteriophage source:
   (1) Bacteriophages and strains: MS2 Source: ATCC #15597-B1
   (2) φX174 Source: ATCC #13706-B1
b) Contact Time: 15 minutes.
c) Organic Load: 5% serum in viral inoculum.
d) Plaque assay. Three technical replicates were run per formulation.

### Results

As shown in Table 4, all three formulations showed ˜1 log reduction against the enveloped bacteriophage (φX174). Surprisingly, Bovine colostrum lactoferrin exhibited the highest antiviral activity against the non-enveloped bacteriophage MS2 with a 4-log reduction, while the other two formulations exhibited about a 1-log reduction or less. A 1 log reduction corresponds to inactivation of ˜90% of starting inoculum. A 4-log reduction corresponds to inactivation of ˜99% of starting inoculum.

TABLE 4
Antiviral Activity of Nasal Spray

| | φX174 (enveloped) | MS2 (non-enveloped) |
|---|---|---|
| | Log10 reduction | Log10 reduction |
| Ingredient | (±SD)/Viral kill rate | (±SD)/Viral kill rate |
| Fucoidan 0.1% in | 0.97 log decline (±0.02) | 0.44 log decline (±0.04) |
| BKC 0.1% | 90% kill rate in 15 | 80% kill rate in 15 |
| | minutes | minutes |
| Lactoferrin from | 0.99 log decline (±0.07) | 4.02 log decline (±0.16) |
| bovine colostrum | 90% kill rate in 15 | 99.99% kill rate in 15 |
| 0.1% in BKC 0.1% | minutes | minutes |
| Fucoidan 0.1% + | 1.01 log decline (±0.07) | 0.50 log decline (±0.17) |
| Lactoferrin from | 90% kill rate in 15 | 80% kill rate in 15 |
| bovine colostrum | minutes | minutes |
| 0.1% in BKC 0.1% | | |

SD = standard deviation Log₁₀ reduction

Thus, as shown, compositions comprising colostrum, are 90% effective at killing enveloped viruses, and 99.9% effective at killing of non-enveloped viruses. Addition of an algan sulphated polysaccharide (e.g., fucoidan) may provide additional benefit in that it provides a physical barrier to cold and flu viruses in the nasal canal.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

What is claimed is:

1. A method for inhibiting, halting, or slowing replication of a virus, comprising administering to a nasal cavity of a subject in need thereof a composition comprising colostrum.

2. The method of claim 1, wherein the composition is in the form of a nasal spray, nasal drops, nasal mist or a nasal swab.

3. The method of claim 1, wherein the colostrum is a bovine colostrum.

4. The method of claim 1, wherein the virus is an airborne respiratory virus.

5. The method of claim 1, wherein the virus is a non-enveloped virus.

6. The method of claim 5, wherein the non-enveloped virus is selected from the group consisting of rhinovirus, human rhinovirus (HRV), viruses of the family picornaviridae, viruses of the family adenoiridae, adenovirus, adeno-associated virus, parvovirus, bocavirus (BoV), and human bocavirus (HBoV).

7. The method of claim 1, wherein the virus is an enveloped virus.

8. The method of claim 7, wherein the enveloped virus is selected from the group consisting of influenza virus, orthomyxoviruses, coronavirus, human coronavirus (HCoV), severe acute respiratory syndrome (SARS) coronavirus (SARS-CoV), SARS-CoV-2 (COVID-19), viruses of the family paramyxoviridae, respiratory syncytial virus (RSV), human respiratory syncytial virus (HRSV)), parainfluenza virus (PIV), human parainfluenza virus (HPIV), pneumovirus (PMV), human pneumovirus (HPMV), metapneumovirus (MPV), human metapneumovirus (HMPV)), respirovirus, and rubulavirus.

9. The method of claim 1, wherein the composition comprises from about 0.01% to about 10% by weight of colostrum.

10. The method of claim 1, wherein the composition further comprises at least one excipient capable of creating an ionic charge.

11. The method of claim 10, wherein the excipient is an organic quaternary ammonium compound that creates an electrostatic field.

12. The method of claim 11, wherein the organic quaternary ammonium compound is benzalkonium chloride or docosyltrimethylammonium chloride.

13. The method of claim 1, wherein the composition further comprises at least one excipient selected from the group consisting of a solvent, a thickening agent, a plasticizer, a preservative, an absorption or permeability enhancing agent, a conditioner, an emulsifier, a surfactant, an antioxidant, a buffering agent, an emollient, a humectant, a suspending agent, an opacifier, a propellant, and a binder.

14. The method of claim 1, wherein the composition further comprises algae or an extract from algae.

15. The method of claim 14, wherein the algae are seaweed algae selected from the group consisting of brown algae, red algae and green algae.

16. The method of claim 1, wherein the composition further comprises an algan polysaccharide.

17. The method of claim 14, wherein the algan polysaccharide is selected from the group consisting of fucoidan derived or isolated from brown algae, carrageenan derived or isolated from red algae, and ulvan derived or isolated from green algae.

18. The method of claim 16, wherein the algan polysaccharide is a fucoidan derived or isolated from brown algae selected from the group consisting of Analipus, Chorda, Dictoyta, Fucus, Kjellmaniella, Pelvetia, Sargassum, and Undaria.

19. The method of claim 14, wherein the composition comprises from about 0.01% to about 10% by weight of the algan polysaccharide.

20. The method of claim 1, wherein the composition is at least 90% effective at killing virus particles.

21. The method of claim 1, wherein the composition is at least 99% effective at killing virus particles.

22. The method of claim 1, wherein the subject has a viral infection selected from the group consisting of upper respiratory infection (URI), bronchiolitis, croup, bronchitis, pneumonia, asthma, asthma exacerbation, chronic obstructive pulmonary disease (COPD), COPD exacerbation, pharyngoconjunctival fever (PCF), and severe acute respiratory syndrome (SARS).

23. A method for treating a viral infection, comprising administering to a nasal cavity of a subject in need thereof a composition comprising colostrum.

24. The method of claim 23, wherein the viral infection is caused by an airborne respiratory virus.

25. The method of claim 24, wherein the composition inhibits, halts or slows replication of the airborne respiratory virus.

26. The method of claim 23, wherein the composition further comprises an algan polysaccharide.
